(19) United States
(12) Patent Application Publication
(10) Pub. No.: US 2019/0206546 A1
(43) Pub. Date: Jul. 4, 2019

(54) MODELING AND LEARNING CHARACTER TRAITS AND MEDICAL CONDITION BASED ON 3D FACIAL FEATURES

(71) Applicant: Dirk Schneemann, LLC, Malibu, CA (US)

(72) Inventor: Dirk Schneemann, Malibu, CA (US)

(21) Appl. No.: 16/296,072

(22) Filed: Mar. 7, 2019

Related U.S. Application Data
(63) Continuation of application No. 15/860,395, filed on Jan. 2, 2018.

(51) Int. Cl. G16H 30/40; G06K 9/00; A61B 5/00; G06K 9/32; G06K 9/46; G06K 9/62; A61B 5/11; A61B 5/16; A61B 5/055; A61B 5/107; G06K 9/66; G06T 7/11; G06T 7/00; G16H 50/20; G06N 3/08; G06N 3/04

(52) U.S. Cl. CPC G16H 30/40; A61B 6/032; A61B 5/7282; G06K 9/3233; G06K 9/4628; G06K 9/627; G06K 9/00302; A61B 5/11; A61B 5/0075; A61B 5/167; G06K 9/00214; G06K 9/00275; A61B 5/055; A61B 5/7267; A61B 5/1077; A61B 5/7275; G06K 9/66; G06T 7/11; G06T 7/0016; A61B 5/0077; G16H 50/20; G06N 3/084; G06N 3/0454; A61B 5/7485; G06K 9/00281; A61B 5/1103; A61B 2576/02; A61B 5/163; G06K 9/00228

(57) ABSTRACT

A computer-implemented method for identifying character traits associated with a target subject includes acquiring image data of a target subject from an image data source, rendering a 3D image data set, comparing each of a plurality of regions of interest within the 3D image set to a historical image data set to identify active regions of interest, grouping subsets of the regions of interest into one or more convolutional feature layers, wherein each convolutional feature layer probabilistically maps to a pre-identified character trait, and applying a convolutional neural network model to the convolutional feature layers to identify a pattern of active regions of interest within each convolutional feature layer to predict whether a target subject possesses the pre-identified character trait.

### CROSS-REFERENCING TO RELATED APPLICATIONS

This present application is a continuation of U.S. patent application Ser. No. 15/860,395, filed on Jan. 2, 2018, which claims the benefit to U.S. Provisional Patent Application No. 62/440,574, filed on Dec. 30, 2016, which are incorporated herein by reference in their entirety.

### TECHNICAL FIELD

The disclosed technology relates generally to applications for identifying character traits and medical condition of a target subject, and more particularly, some embodiments relate to systems and methods modeling and learning character traits based on 3D facial features and expressions.

### BACKGROUND

Facial recognition technology has become more widely applications other than simple identification of a target subject. In some applications, analysis of facial features may be used to determine personality traits for an individual. In particular, studies have focused on determining personality traits using analysis of facial and body expressions, and “body language,” including gestures and gesticulations. For example, some research suggests that the shape of the nasal root supplies statements about the life zone with the expression of spiritual impulses in the interaction with other people, the energy use becomes apparent at the temples, the forehead regions express spiritual activity, the upper forehead allows recognition of goodwill and affection and the chin and lower jaw provide information on motivation and assertiveness.

Methods for determining personality traits based on facial recognition algorithms generally rely on the assumption that specific character traits can be learned directly from an input space either by Support Vector Machine (SVM) or Hidden Markov Model (HMM) approaches. These approaches are generally prohibitively inefficient for analyzing large and complex datasets. For example, SVM and HMM approaches struggle with analysis of high definition, high speed, and/or high pixel depth datasets which may be used to identify multiple granular features, facial textures, 3D features, and/or saliency across multiple facial features. Thus, where SVM or HMM based techniques may be applied to small datasets, for example, to compare captured data from a target subject against predetermined or hardcoded reference datasets, the SVM and HMM algorithms do not scale up with larger data sets, e.g., comprising thousands of images. Moreover, available personality trait recognition systems and methods tend to be limited, not only to smaller data sets, but also to small and discrete result sets that may only include a few (e.g., tens) of personality traits. In the medical field, researchers developed systems for predicting age-related macular degeneration from visual features extracted from the retina, e.g., to predict whether skin lesions are cancerous.

### BRIEF SUMMARY OF EMBODIMENTS

According to various embodiments of the disclosed technology, systems and methods for modeling and learning character traits based on 3D facial features may include applying a convolutional neural network learning algorithm to an image data set to identify a correlation to one or more character traits or medical conditions. By applying the convolutional neural network learning model to multiple regions of interest within the image data set, a more granular analysis may be achieved across a large number of possible character traits with higher specificity than is possible with previous SVM and HMM based models.

Another feature of the convolutional neural network model is its ability to learn through tuning by evaluating different sets of regions of interest available in the image data set (e.g., different specific features of interest on a target subject's face), and then adjusting the model based on comparison with historical data, data acquired by other diagnostic tools, or user input. Patterns may be detected across groups of regions of interest, wherein each region of interest group may be applied as a convolutional feature layer within the convolutional neural network model. Patterns detected by the convolutional neural network model may then be correlated with specific character traits or medical conditions, and the results may be tuned via supervised learning using user feedback.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1A illustrates an example system for modeling and learning character traits based on 3D facial features, consistent with embodiments disclosed herein.

FIG. 1B illustrates an example image data set for modeling and learning character traits of a target subject, consistent with embodiments disclosed herein.

FIG. 1C is a flow chart illustrating an example method for acquiring and processing image data sets for modeling and learning character traits, consistent with embodiments disclosed herein.

FIG. 2 is a flow chart illustrating an example method for acquiring and processing image data sets for modeling character traits, consistent with embodiments disclosed herein.

FIG. 3 illustrates an example method of processing and learning from image data sets using a convolutional neural networks, consistent with embodiments disclosed herein.

FIG. 4 is a flow chart illustrating an example method for processing and learning from image data sets using convolutional neural networks, consistent with embodiments disclosed herein.

FIG. 5 is a flow chart illustrating an example method for acquiring and processing 3D image data sets for to identify medical or health related information about a target subject, consistent with embodiments disclosed herein.

FIG. 6 illustrates an example system for identifying medical or health related information about a target subject, consistent with embodiments disclosed herein.

FIG. 7 illustrates an example system for identifying character traits about a target subject using feedback data from a remote data source, consistent with embodiments disclosed herein.

FIG. 8 illustrates an example system for identifying character traits about a target subject using a mobile acquisition device and feedback data from other sources such as other users with mobile devices, consistent with embodiments disclosed herein.

FIG. 9 illustrates an example computing engine that may be used in implementing various features of embodiments of the disclosed technology.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION OF THE EMBODIMENTS

The technology disclosed herein is directed toward a system and method for identifying character traits using facial and expression recognition to analyze image data sets. Embodiments disclosed herein incorporate the use of a convolutional neural network algorithm and a learning feedback loop to correlate the image data sets to a database of character traits, inclusive of medical conditions, and to learn based on historical data or user feedback.

More specifically, examples of the disclosed technology include acquiring image data of a target subject from one or more image data sources, rendering or acquiring a 3D image data set, comparing a plurality of regions of interest within the 3D image set to historical image data to determine the presence of features within each of the plurality of regions of interest, grouping subsets of the regions of interest into one or more convolutional feature layers, wherein each convolutional feature layer probabilistically maps to a pre-identified character trait, and applying a convolutional neural network algorithm to identify whether the target subject possesses the pre-identified character trait.

Some embodiments further include training the convolutional neural network using feedback input through a user interface. In some examples, the character traits may include medical conditions. The regions of interest may relate to features detected on a target subject's head or face, and may further include expressions detected using video or time-sequenced image data.

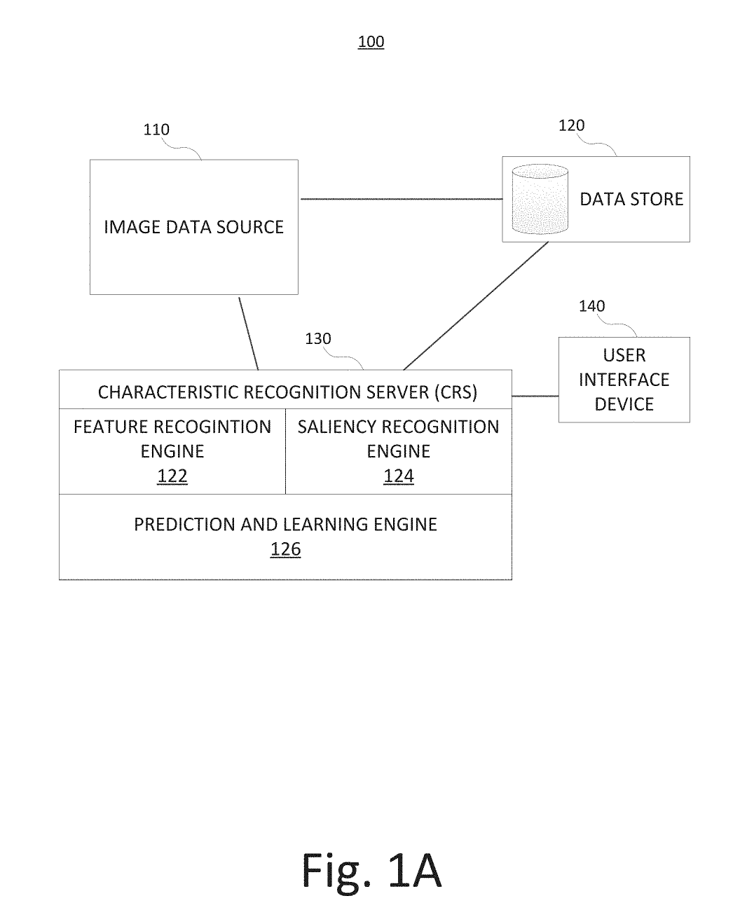

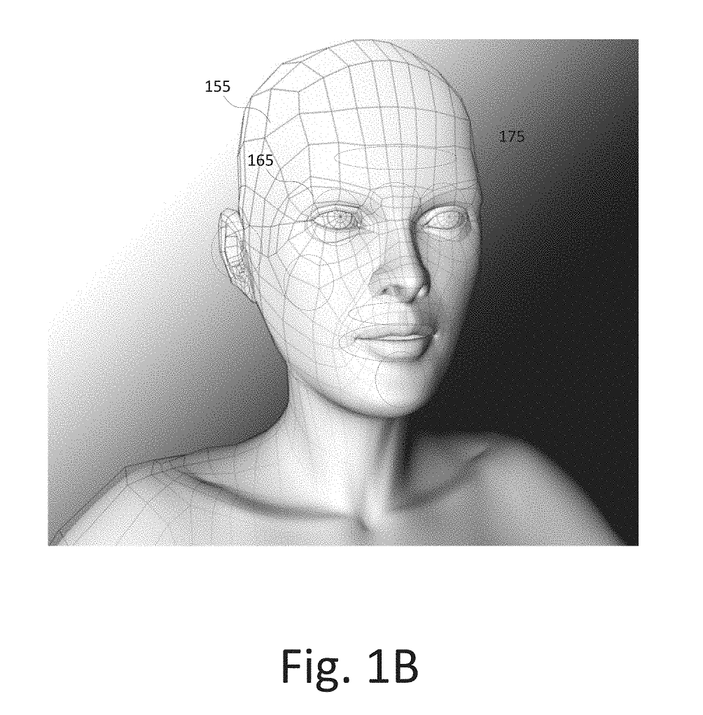

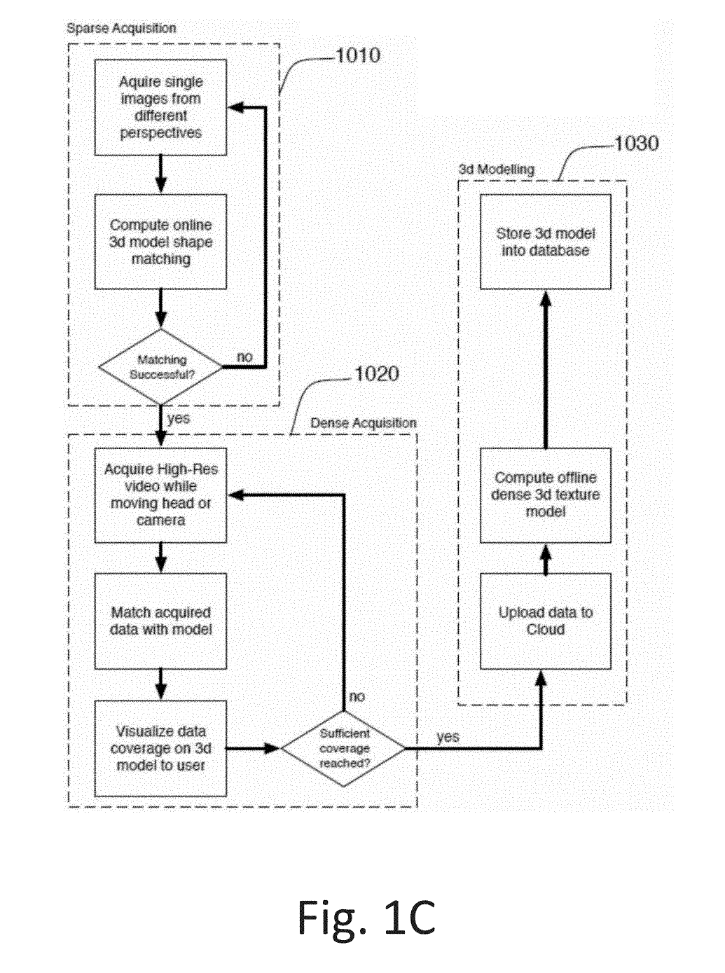

FIG. 1A illustrates an example system for modeling and learning character traits based on 3D facial features. Referring to FIG. 1A, system for modeling and learning character traits based on 3D facial features and/or texture data 100 includes an image data source 110. Image data source 110 may be a still, video, a standard definition, a high definition, ultrahigh definition, infrared, 3D point cloud data, or other digital or analog camera as known in the art. Image data source 110 may also include laser scanner, CAT scanner, MRI scanner, ultrasound scanner, or other detection devices capable of imaging anatomical features or objects either by texture or 3D shape. Some examples, image data source 110 may be a mobile phone camera. Image data source 110 may also include an image data store such as a picture archive or historical database. In some examples, image data source 110 may include multiple imaging devices, such that imaging data from different sources may be combined. For example, imaging data from a high-definition or ultrahigh definition video camera may be combined with imaging data from a still camera, laser scanner, or medical imaging device such as a CAT scanner, ultrasound scanner, or MRI scanner. Image data source 110 is configured to acquire imaging data from a target subject. For example, the target subject or subjects may include a human face, a human body, an animal face, or an animal body.

Image data source 110 may be communicatively coupled to characteristic recognition server (CRS) 130. For example, CRS 130 may be direct attached to image data source 110. Alternatively, image data source 110 may communicate with CRS 130 using wireless, local area network, or wide area network technologies. In some examples, image data source 110 may be configured to store data locally on a removable data storage device, and data from the mobile data storage device may then be transferred or uploaded to CRS 130.

CRS 130 may include one or more processors and one or more non-transitory computer readable media with software embedded thereon, where the software is configured to perform various characteristic recognition functions as disclosed herein. For example, CRS 130 may include feature recognition engine 122. Feature recognition engine 122 may be configured to receive imaging data from image data source 110, and render 3D models of the target subject. Feature recognition engine 122 may further be configured to identify spatial patterns specific to the target subject. For example, feature recognition engine 122 may be configured to examine one or more regions of interest on the target subject, compare the image data and/or 3D render data from those regions of interest with spatial data stored in data store 120, to determine if known patterns stored data store 120 match patterns identified in the examined regions of interest from the acquired image data set.

CRS 130 may also include a saliency recognition engine 124. Saliency recognition engine 124 may be configured to receive video image data, 3D point clouds, or still frame time sequence data from image data source 110. Similar to feature recognition engine 122, saliency recognition engine 124 may be configured to examine one or more regions of interest on the target subject, and identify specific movement patterns within the image data set. For example, saliency recognition engine 124 may be configured to identify twitches, expressions, eye blinks, brow raises, or other types of movement patterns which may be specific to a target subject.

Historical data sets of both still frame image data and saliency data may be stored in data store 120. Data store 120 may be direct attached to CRS 130. Alternatively, data store 120 may be network attached or located in a storage area network, in the cloud-based, or otherwise communicatively coupled to CRS 130, and/or image data source 110.

CRS 130 may also include a prediction and learning engine 126. Prediction and learning engine 126 may be configured to predict characteristics specific to the target subject based on patterns identified by feature recognition engine 122 and/or saliency recognition engine 124 using prediction algorithms as disclosed herein. The prediction algorithms, for example, may include Bayesian algorithms to determine the probability that a specific character trait is associated with a region of interest, or pattern of multiple regions of interest within image data taken of a target subject and which correlate to a particular character trait. Prediction and learning engine 126 may be configured to adapt and learn. For example, a first prediction of a first character trait may be identified to be associated with the target subject. A user, using user interface device 140, may evaluate the accuracy of the first prediction, and determined that the prediction was incorrect. Using a characteristic identified by the user, or a second prediction, prediction and learning engine 126 may identify a second character trait that is likely associated with the target subject. Upon confirmation that the second prediction is accurate, prediction and learning engine 126 may update a historical database of predictions and associated feature and/or saliency patterns identified within one or more regions of interest in the image data set, as stored in data store 120.

FIG. 1B illustrates an example image data set for modeling and learning character traits of a target subject. Example image data set 155 may be a rendered 3D representation of the target subject combined with texture/color information (not shown in the figure). As illustrated, multiple regions of interest (e.g., regions of interest 165 and 175) may be predefined in data store 120 or using user interface 140, and/or learned by prediction and learning engine 126. Regions of interest may be selected based on the propensity for reflecting specific behavioral, personality, or other character traits of the target subject. A region of interest may be examined by either feature recognition engine 122 or saliency recognition engine 124. Prediction and learning engine 126 may analyze pattern matches identified by feature recognition engine 122 and saliency recognition is 124 across multiple regions of interest to identify specific patterns which correlate to known character traits or medical condition. For example, the target subject may display a specific raise of the brow, sigh, and squint of the eye, all at the same time, which may match a pattern which correlates to a character trait (e.g., an introverted or extroverted personality, stress, personality disorder, medical condition, etc.). The system may also identify correlations between two static areas of interest without any movement considerations. Static areas can be described by color and 3D shape. For example, static 3D shapes can be the geometry of facial landmarks such as nose, ears, chin, cheeks. Static color areas can be the coloring and texture from pimples, dents, bumps, folds, and wrinkles in the face.

FIG. 1C is a flow chart illustrating an example method for acquiring and processing image data sets for modeling and learning character traits. The example method illustrated in FIG. 1C may assure that a sufficient amount of high resolution image data is acquired to generate a dense 3D texture map sufficient to evaluate features within one or more desired regions of interest, while not acquiring too much image data as to overburden the system and data storage. In some examples, the example method may include: 1) associating sparse image data with a rough 3D model; and 2) ensuring all regions are sufficiently covered with high resolution data.

Referring to FIG. 1C, an example method for acquiring and processing image data sets may include a sparse acquisition process 1010, intense acquisition process 1020, and a 3D modeling process 1030. For example, sparse acquisition process 1010 and dense acquisition process 1020 may be performed by image data source 110 and feature recognition engine 122. 3D modeling process 1030 may be performed by feature recognition engine 122. Sparse acquisition process 1010 may include acquiring single images from different perspectives, computing online 3D model shape matching (using feature recognition engine 122), determining whether matching was successful. If matching is unsuccessful, the process may include acquiring more images from the same or different perspectives.

If matching is successful (e.g., specific features within regions of interests of the target subject are identified), the method may further include dense acquisition process 1020. Dense acquisition process 1020 may include acquiring high-resolution video while moving the camera, or alternatively, while the target subject moves or turns his/her head. Dense acquisition process 1020 may further include matching the acquired data with a model stored in data store 120 using saliency recognition engine 124. User may visualize the data coverage on the 3D model via user interface 140 to determine if the rendered image data sufficiently covers the model. In some examples, saliency prediction engine 124 may automatically evaluate whether the image data sufficiently covers the model using automated 3D rendering techniques as known in the art. If the image data coverage is insufficient, then more high-resolution video may be acquired.

If sufficient image data exists to cover the model, at least across desired regions of interest, then the method may further include 3D modeling at step 1030. 3D modeling may include computing a 3D detection model and storing the model in a database, for example, located on data store 120. The dense 3D texture modeling may be performed by saliency recognition engine 124, or may be accomplished using an off-line 3D rendering system or a cloud-based rendering system.

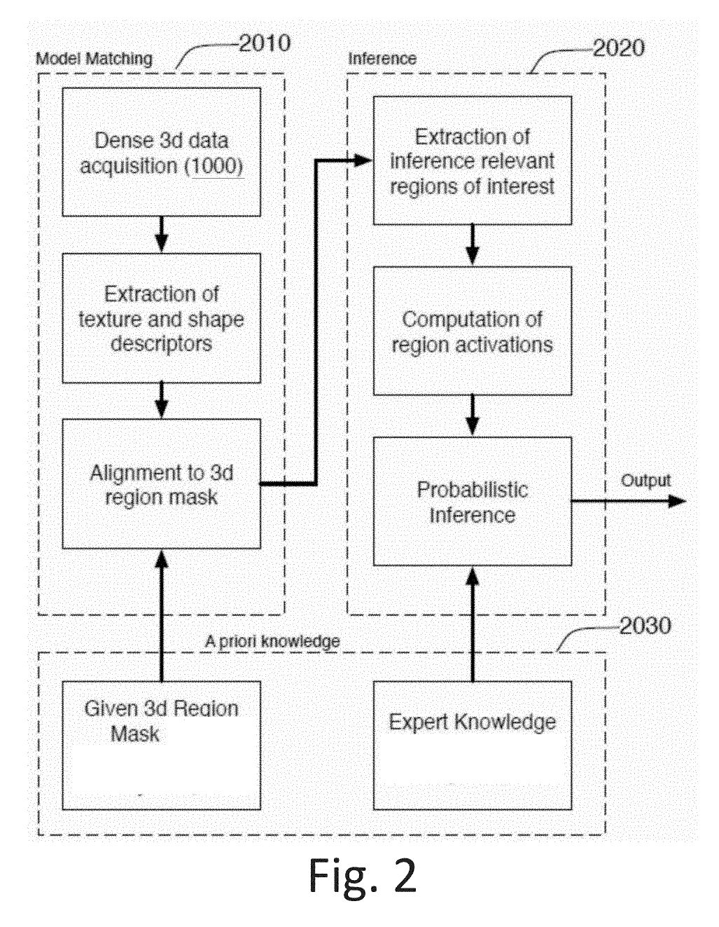

FIG. 2 is a flow chart illustrating an example method for acquiring and processing image data sets for modeling and learning character traits. Referring to FIG. 2, a method for acquiring and processing image data sets may further include a model matching process 2010, an inference process 2020, and a comparison process with historical data at step 2030. Model matching process 2010 may include receiving dense 3D data, for example from dense acquisition process 1020, extraction of texture and shape descriptors, and alignment to a 3D region mask using spatial pattern matching techniques as known in the art. In some examples, a user may assist in the alignment of the dense 3D data set to the 3D region mask process through user interface 140.

Inference process 2020 may include extraction of inference relevant regions of interest, computation of region activations, and a probabilistic inference, e.g., using prediction and learning engine 126. In some examples, prediction and learning engine 126 may use a Bayesian reasoning algorithm. For example, the region activations may reflect specific modeled 3D image data within identified regions of interest which match historic 3D image data from data store 120 for the same regions of interest which correlate to previously identified character traits. In some examples, multiple regions of interest will be activated creating a pattern of region activations. The probabilistic inference may be a weighted value identifying a likely correlation between the pattern of region activations and specific character traits. The probabilistic inference may be initially seeded by a user through user interface 140 (e.g., using expert knowledge or historical data), or by a predetermined or historical weighting.

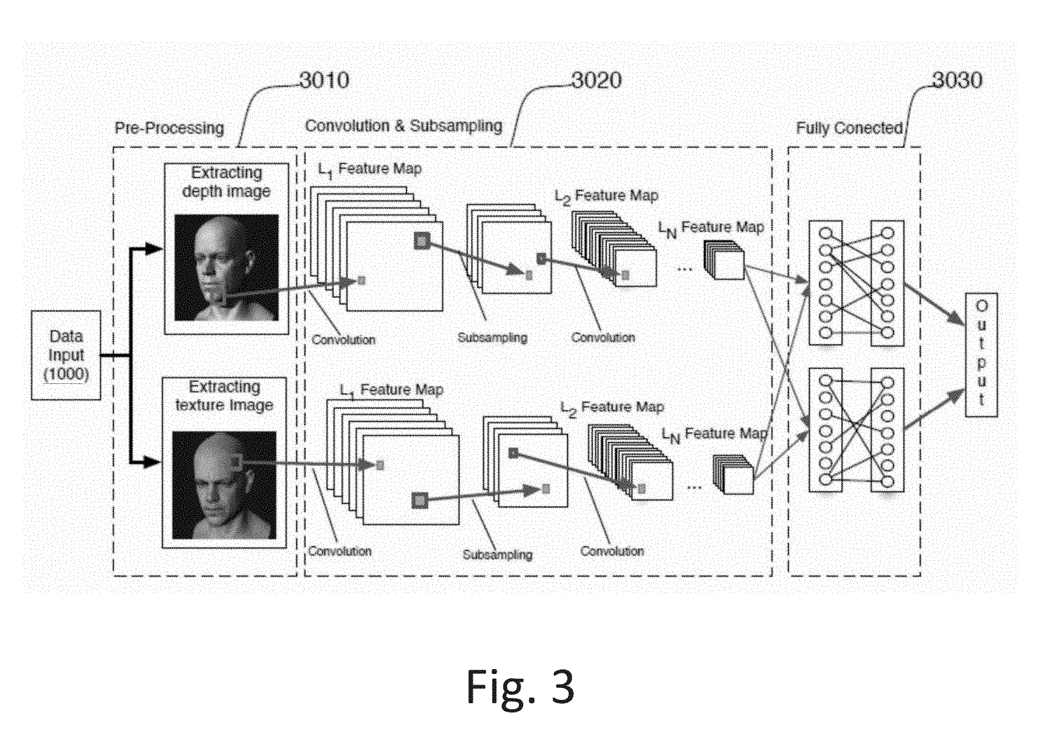

FIG. 3 illustrates an example method of acquiring processing image data sets using a convolutional neural networks. Referring to FIG. 3, a preprocessing algorithm 3010 may be applied to imaging data acquired from image data source 110 prior to identifying region activations, for example, an inference process 2020 referenced in FIG. 2. Preprocessing algorithm 3010 may include extracting a depth image based on shadowing or detection of structures from motion, as detected in the image data set to identify features in all three spatial dimensions. Preprocessing algorithm 3010 may also include extracting texture image data, for example, to identify hair, whiskers, eyebrows, pock marks, rough skin, wrinkles, or other textural elements present on a target subject's face.

The method may further include convolution and subsampling process 3020. In some examples, convolution and subsampling process 3020 includes identifying one or more convolutional layers. For example, in the context of facial feature and expression recognition, a convolutional layer may include a set of regions of interest which, if activated by matching them to data acquired from the target subject, may be correlated with a specific character trait. For example, mouth movement, brow movement, and eye lid movement may together comprise an example convolutional feature layer which may be activated if a target subject sighs, raises an eyebrow, and closes his/her eyes at the same time. Detection and identification of static features and dynamic features may be incorporated in the same convolutional feature layer or network. Static features detected by the network may be, for example, color, texture, spatial geometry and size of facial landmarks such as nose, mouth, cheeks, forehead regions, ears, yaw. Color and texture based static features detected by the network can be, for example, wrinkles, bumps, dents and folds. Multiple convolutional layers may be analyzed across a single image data set in a manner consistent with convolutional neural network analysis.

As illustrated in FIG. 3, depth image data and/or texture image data from the image data model may be applied to an L-1 convolutional feature map. Data from the L-1 convolutional feature map may then be subsampled and applied to an L-2 convolutional feature map, and the process may be repeated through N layers.

By sampling each nested convolutional layer, facial features are composed by combining several feature maps from lower levels. For example, the facial feature of strong cheek bones may be composed of several low level features such as specific color combinations and combinations of geometrical primitives and 3D surface arrangements. Each final feature map in the L-N layer may be associated with one or more facial feature such as spatial geometry and texture of facial regions and landmarks. Within the Fully Connected Layer, combinations of feature maps of the L-N layer may be associated with one or more character traits, such as personality, behavior, and medical condition. For example, a particular personality trait or medical condition may be detected only when a combination of underlying dependent convolutional layers are activated. The activation of a convolutional layer may correspond to all of the regions of interest within that convolutional layer being activated. A region of interest may also be associated with more than one convolutional layer, and convolutional layers may themselves be evaluated and sub sampled in different orders. Inclusion or exclusion of a particular region of interest within any one of the convolutional layers may be determined through a supervised learning process by comparing output from the convolutional neural network process, e.g., at step 3030, with historical data stored in data store 140. Alternatively, a user may adjust the convolutional neural network process by tuning which regions of interest should be applied in which convolutional layers, in the order in which the convolutional layers themselves should be applied. The process of tuning the convolutional neural network by comparing with historical data, or input from a user, is known as training or supervised learning.

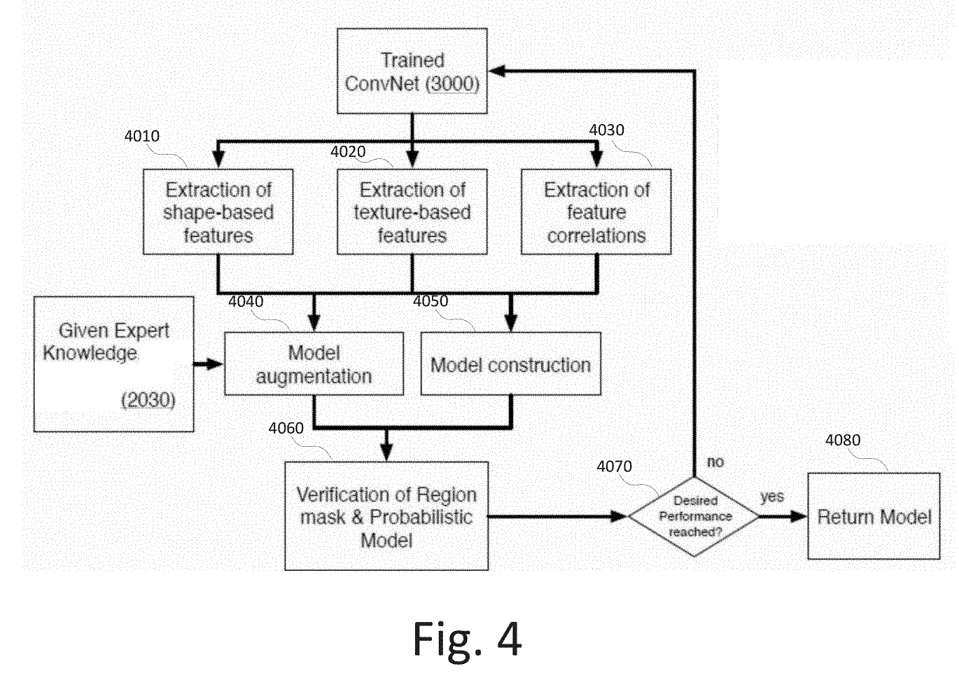

FIG. 4 is a flow chart illustrating an example method for building a classifier model from a trained convolutional neural network. Specifically, the method includes extracting new features from learned convolutional neural networks comprising hierarchal arrangements of convolutional feature layers that are relevant to a classification of the underlying image data set to one or more character traits. Specifically, features may be extracted and added to region masks (2030), which are then applied to a probabilistic inference (2020). In some examples, the method may include: (1) augmenting an existing model (region mask and rules); and (2) creating a new model (region mask and rules) if no historical data is available.

Referring to FIG. 4, trained convolutional neural network data from step 3030, as referred to in FIG. 3, may be processed by extracting 3D shape-based features at step 4010, extracting texture-based features at step 4020, and/or extracting feature correlations (e.g., relationships between two or more regions of interest as correlated with a particular character trait) at step 4030. The extracted data may then be applied to a 3D model augmentation process at step 4040 or a three model construction process at step 4050 as known in the art of 3D rendering. A user may interact with the 3D rendering process using user interface 140. The verification of the region mask and probabilistic inference data at step 4060 may include determining the activation of specific convolutional layers within the applied convolutional neural network and weighting the correlation of that data to possible sets of character traits using probabilistic coefficients, then tuning the convolutional neural network (e.g., by adding or removing regions of interest from convolutional layers, or changing the order that the convolutional layers are applied) to determine which convolutional neural networks highly correlate with which character traits or medical condition.

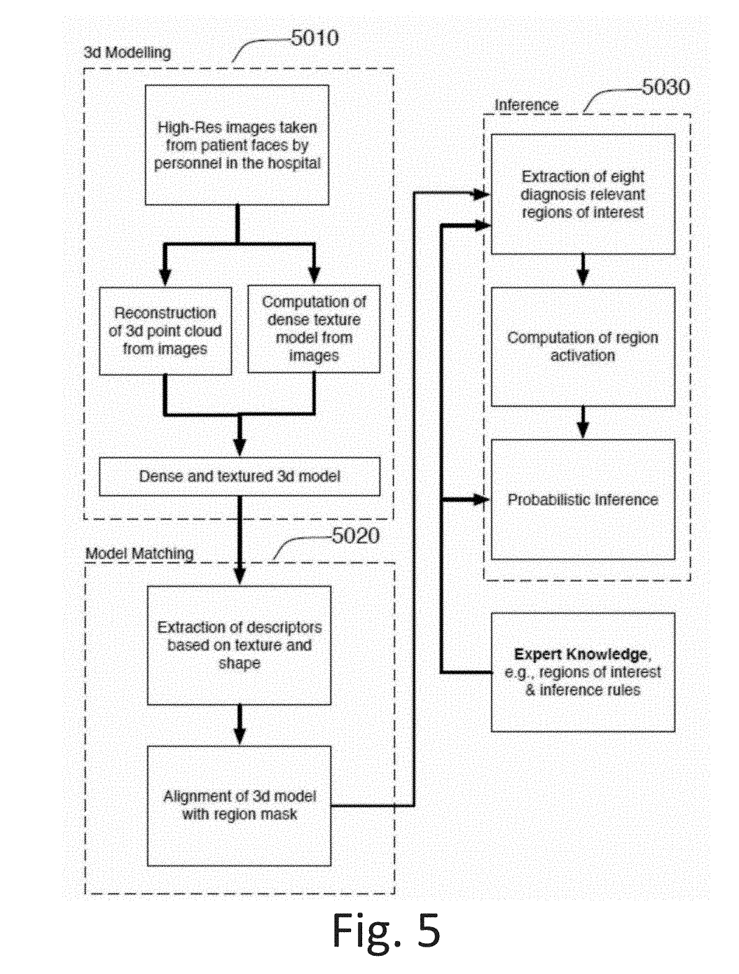

FIG. 5 is a flow chart illustrating an example method for acquiring and processing textured 3-D image data sets to identify medical or health related information about a target subject. As illustrated, 3-D modeling process 5010 and model matching process 5020 are similar to steps 2010 and 2020 referred to in FIG. 2. In one example involving medical diagnosis, extraction of a small subset of diagnosis relevant regions of interest may be useful. In one such embodiment, the diagnosis relevant regions of interest may be used. Diagnosis regions of interest may be based on historical data, e.g., as stored in a database, or expert knowledge. Inference step 5030 that includes computation of region activation and application of a probabilistic inference, using the convolutional neural network process referred to in FIG. 3. Correlation of activated convolutional layers across, for example, eight or more diagnosis relevant regions of interest may then be correlated with a set of medical conditions. The resulting data may be correlated with historical diagnosis data taken using other methods, for example, evaluation by a medical professional using medical diagnostic equipment. The convolutional neural network for medical diagnosis may then be tuned as described above with reference to FIG. 3 to correlate the activated medical diagnosis relevant convolutional layers to individual medical conditions. After the system learns, the trained convolutional neural network may be applied to an image data set from a target subject to assist in identification of the target subject's individual medical conditions.

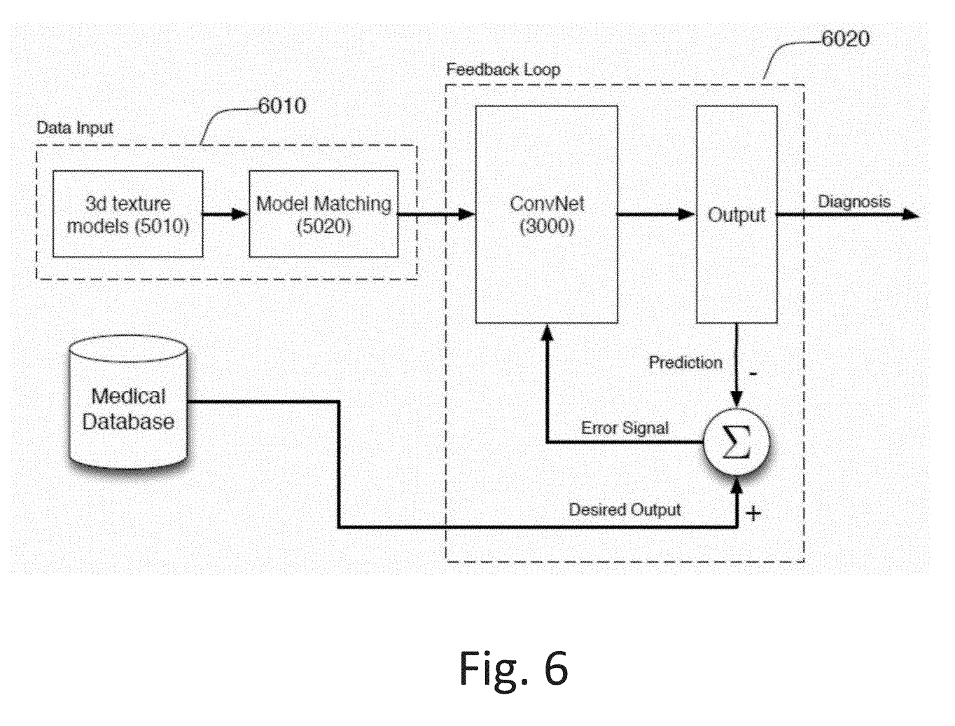

FIG. 6 illustrates an example system for training the convolutional neural network referenced in FIG. 3 to identify medical or health related information about a target subject. As illustrated, a feedback loop 6020 may be applied to data output from 3D texture modeling process 5010 and model matching process 5020 to tune the convolutional neural network. For example, output data from the convolutional neural network algorithm 3020 may be used to generated a predicted diagnosis. If the prediction is not accurate as compared with desired output from a medical database, for example, as stored on data store 120, then an error signal may be generated and used to fine tune the convolutional neural network process. For example, a specific convolutional layer that may probabilistically be likely to correlate to a specific medical condition may be added to the convolutional neural network, or the order of application of convolutional layers may be adjusted. Probabilistic weightings for each convolutional layer may also be adjusted to indicate, for example, a higher likelihood that a specific convolutional layer would be present in relation to a specific medical condition as opposed to a convolutional layer that is only sometimes present in relation to a specific medical condition.

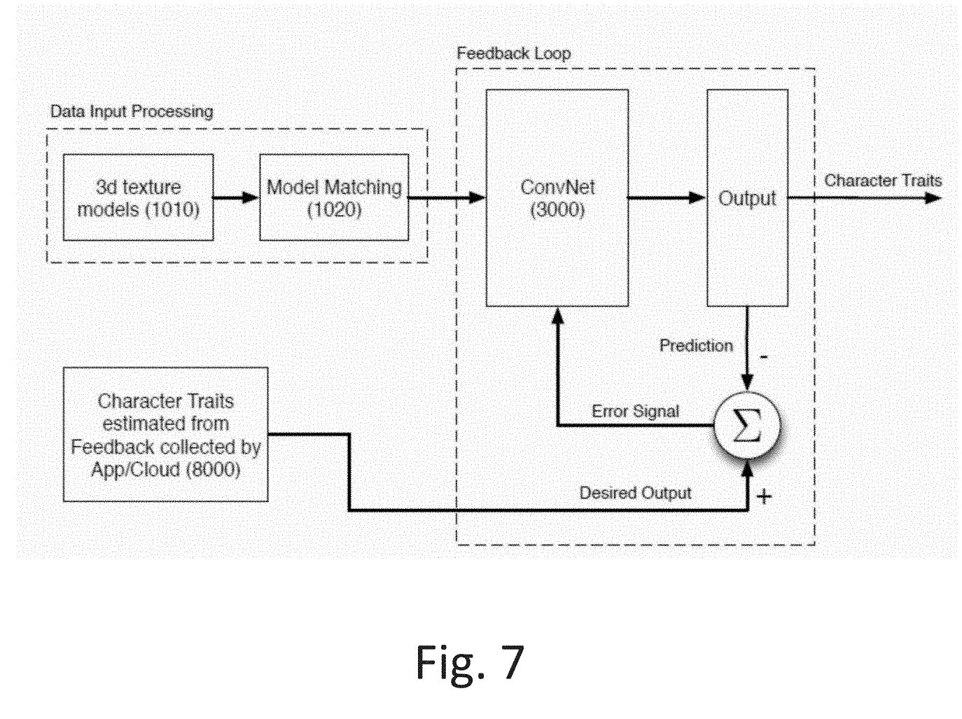

FIG. 7 illustrates an example system for training the convolutional neural network referenced in FIG. 3 to identify character traits of a target subject using feedback data from a remote data source, similar to the learning process described above with respect to FIG. 6. Experiments have identified several key groupings of regions of interest on human facial anatomy that correlate to specific observable character traits. These regions of interest, or zones, may be applied to one or more convolutional layers in the convolutional neural network in order to identify

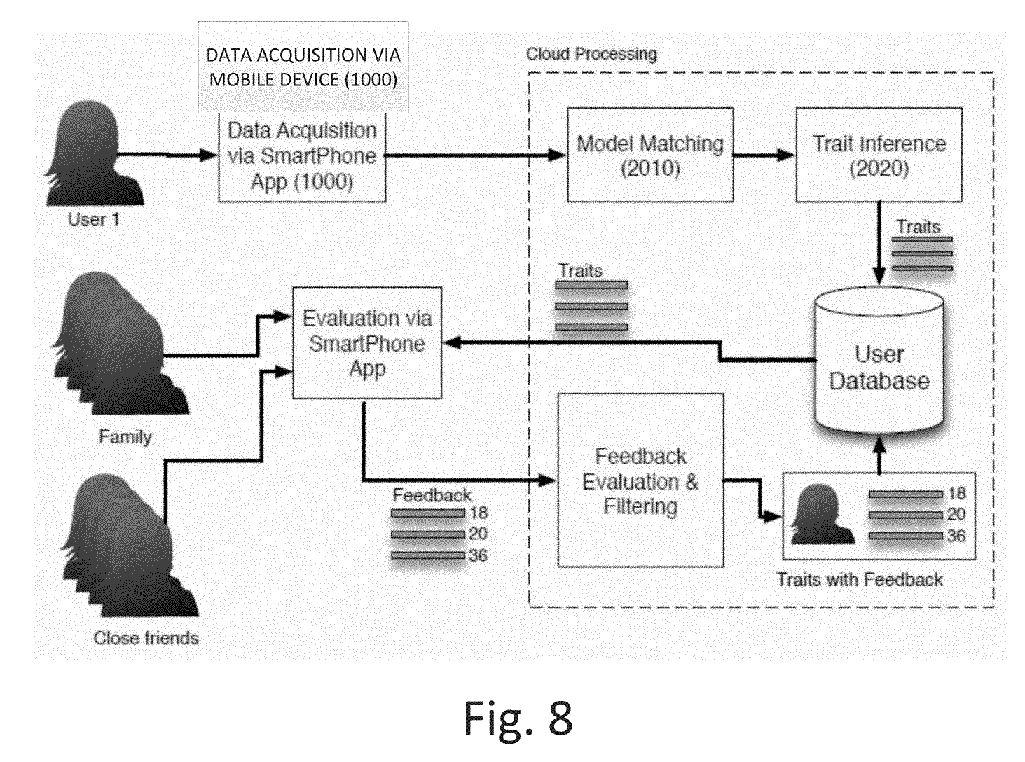

FIG. 8 illustrates an example system for identifying character traits about a target subject using a mobile acquisition device and feedback data from a remote data source. For example, image data source 110 may be a mobile device, such as a smart phone. A user may acquire image data using the smart phone camera and upload the data, using a wireless or cellular network, to either a local or a remote CRS 130. In the case of a remote or cloud-based CRS 130, model matching step 2010 and inference step 2020, as well as application of the convolutional neural network algorithm 3020, may be accomplished using a cloud-based server. Results may be stored on a user database, for example in data store 120, which may also be located in the cloud. Identified character traits may be made available for evaluation through user interface 140, which for example, may be another mobile device, such as a family member's or friend's smart phone. A mobile device based app may then be used to evaluate the data and apply feedback to the convolutional neural network in order to train the convolutional neural network. Historical data across individual target subjects, as well as compilations of multiple target subjects, may be stored in the user database and used to tune the overall accuracy of the convolutional neural network in order to more accurately identify character traits based on uploaded image data sets.

In some embodiments, if an image data set is insufficient to indicate all required regions of interest necessary for accurate evaluation by the convolutional neural network (e.g., important regions of interest cannot be visualized or modeled because the image data set is incomplete), an alert may be sent back to the source mobile device via an app to alert the user to acquire additional image data sets.

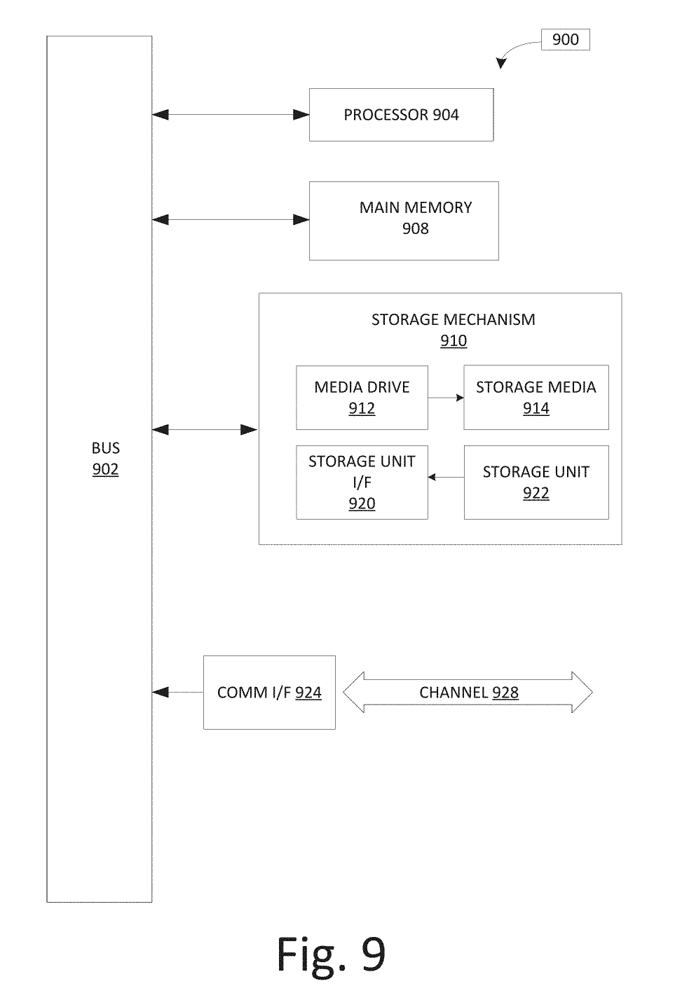

As used herein, the term engine might describe a given unit of functionality that can be performed in accordance with one or more embodiments of the technology disclosed herein. As used herein, an engine might be implemented utilizing any form of hardware, software, or a combination thereof. For example, one or more processors, controllers, ASICs, PLAs, PALs, CPLDs, FPGAs, logical components, software routines or other mechanisms might be implemented to make up a engine. In implementation, the various engines described herein might be implemented as discrete engines or the functions and features described can be shared in part or in total among one or more engines. In other words, as would be apparent to one of ordinary skill in the art after reading this description, the various features and functionality described herein may be implemented in any given application and can be implemented in one or more separate or shared engines in various combinations and permutations. Even though various features or elements of functionality may be individually described or claimed as separate engines, one of ordinary skill in the art will understand that these features and functionality can be shared among one or more common software and hardware elements, and such description shall not require or imply that separate hardware or software components are used to implement such features or functionality.

Where components or engines of the technology are implemented in whole or in part using software, in one embodiment, these software elements can be implemented to operate with a computing or processing engine capable of carrying out the functionality described with respect thereto. One such example computing engine is shown in FIG. 9. Various embodiments are described in terms of this example computing engine 900. After reading this description, it will become apparent to a person skilled in the relevant art how to implement the technology using other computing engines or architectures.

Referring now to FIG. 9, computing engine 900 may represent, for example, computing or processing capabilities found within desktop, laptop and notebook computers; hand-held computing devices (PDA's, smart phones, cell phones, palmtops, etc.); mainframes, supercomputers, workstations or servers; or any other type of special-purpose or general-purpose computing devices as may be desirable or appropriate for a given application or environment. Computing engine 900 might also represent computing capabilities embedded within or otherwise available to a given device. For example, a computing engine might be found in other electronic devices such as, for example, digital cameras, navigation systems, cellular telephones, portable computing devices, modems, routers, WAPs, terminals and other electronic devices that might include some form of processing capability.

Computing engine 900 might include, for example, one or more processors, controllers, control engines, or other processing devices, such as a processor 904. Processor 904 might be implemented using a general-purpose or special-purpose processing engine such as, for example, a microprocessor, controller, or other control logic. In the illustrated example, processor 904 is connected to a bus 902, although any communication medium can be used to facilitate interaction with other components of computing engine 900 or to communicate externally.

Computing engine 900 might also include one or more memory engines, simply referred to herein as main memory 908. For example, preferably random access memory (RAM) or other dynamic memory, might be used for storing information and instructions to be executed by processor 904. Main memory 908 might also be used for storing temporary variables or other intermediate information during execution of instructions to be executed by processor 904. Computing engine 900 might likewise include a read only memory (“ROM”) or other static storage device coupled to bus 902 for storing static information and instructions for processor 904.

The computing engine 900 might also include one or more various forms of information storage mechanism 910, which might include, for example, a media drive 912 and a storage unit interface 920. The media drive 912 might include a drive or other mechanism to support fixed or removable storage media 914. For example, a hard disk drive, a floppy disk drive, a magnetic tape drive, an optical disk drive, a CD or DVD drive (R or RW), or other removable or fixed media drive might be provided. Accordingly, storage media 914 might include, for example, a hard disk, a floppy disk, magnetic tape, cartridge, optical disk, a CD or DVD, or other fixed or removable medium that is read by, written to or accessed by media drive 912. As these examples illustrate, the storage media 914 can include a computer usable storage medium having stored therein computer software or data.

In alternative embodiments, information storage mechanism 190 might include other similar instrumentalities for allowing computer programs or other instructions or data to be loaded into computing engine 900. Such instrumentalities might include, for example, a fixed or removable storage unit 922 and an interface 920. Examples of such storage units 922 and interfaces 920 can include a program cartridge and cartridge interface, a removable memory (for example, a flash memory or other removable memory engine) and memory slot, a PCMCIA slot and card, and other fixed or removable storage units 922 and interfaces 920 that allow software and data to be transferred from the storage unit 922 to computing engine 900.

Computing engine 900 might also include a communications interface 924. Communications interface 924 might be used to allow software and data to be transferred between computing engine 900 and external devices. Examples of communications interface 924 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 924 might typically be carried on signals, which can be electronic, electromagnetic (which includes optical) or other signals capable of being exchanged by a given communications interface 924. These signals might be provided to communications interface 924 via a channel 928. This channel 928 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to media such as, for example, memory 908, storage unit 920, media 914, and channel 928. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing engine 900 to perform features or functions of the disclosed technology as discussed herein.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent engine names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “engine” does not imply that the components or functionality described or claimed as part of the engine are all configured in a common package. Indeed, any or all of the various components of a engine, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

I claim:

1. A computer-implemented method for identifying character traits associated with a target subject, the method comprising:
acquiring image data of a target subject from an image data source;
rendering a colored or textured 3D image data set;
comparing, with a characteristic recognition server, each of a plurality of regions of interest within the 3D image set to a historical image data set to identify active regions of interest;
grouping subsets of the regions of interest into one or more convolutional feature layers, wherein convolutional feature layers probabilistically map to pre-identified character traits; and
applying, with a prediction and learning engine, a convolutional neural network model to the convolutional feature layers to train and identify patterns of active regions of interest within each convolutional feature layer to predict whether a target subject possesses the pre-identified character trait.

2. The computer-implemented method of claim 1, further comprising:
storing the one or more convolutional neural networks; and
for each pre-defined character trait, extrapolating from the one or more convolutional neural networks, one or more regions of interest correlated to the pre-defined character trait.

3. The method of claim 2, wherein the extrapolating one or more regions of interest comprises applying a deep learning algorithm to the one or more convolutional neural networks.

4. The computer-implemented method of claim 1, further comprising obtaining, from a user interface, an indication as to whether the target subject possesses the pre-identified character trait.

5. The computer implemented method of claim 4, further comprising generating an error signal if the prediction as to whether the target subject possesses the pre-identified character trait does not match the indication from the user interface.

6. The computer implemented method of claim 5, further comprising tuning the convolutional neural network model by applying, with the prediction an learning engine, the error signal to the convolutional neural network model.

7. The computer-implemented method of claim 6, wherein the tuning of the convolutional neural network model comprises adjusting a set of probabilistic weightings for one or more convolutional layers, wherein a probabilistic weighting indicates a likelihood that the convolutional layer is included in the convolutional neural network model in relation to a corresponding pre-defined character trait.

8. A computer-implemented method for identifying early signs of diseases from features detected in human faces, the method comprising:
acquiring image data of a target subject from an image data sources;
rendering a colored or textured 3D image data set;
comparing each of a plurality of regions of interest within the 3D image set to a historical data set stored in an Electronic Health Record;
grouping subsets of the regions of interest into one or more convolutional feature layers, wherein convolutional feature layers probabilistically map to one or more medical diagnoses; and
applying a convolutional neural network algorithm to the convolutional feature layers to train and identify a pattern of active regions of interest within each convolutional feature layer to render a medical diagnosis.

9. The method of claim 8, further comprising:
storing a plurality of convolutional neural networks, each convolutional neural network comprising a set of convolutional feature layers and one or more corresponding medical diagnoses; and
for each medical diagnosis, extrapolating from the plurality of convolutional neural networks, one or more regions of interest correlated to the medical diagnosis.

10. The method of claim 9, wherein the extrapolating one or more regions of interest comprises applying a deep learning algorithm to the plurality of convolutional neural networks.

11. A system for identifying character traits associated with a target subject, the system comprising:
a characteristic recognition server, an image data source, a user interface, and a data store, wherein the characteristic recognition server comprises a processor and a non-transitory medium with computer executable instructions embedded thereon, the computer executable instructions configured to cause the processor to:
acquire image data of a target subject from the image data source;
render a textured or colored 3D image data set;
compare each of a plurality of regions of interest within the 3D image set to a historical image data set to identify active regions of interest;
group subsets of the regions of interest into one or more convolutional feature layers, wherein convolutional feature layers probabilistically map to pre-identified character traits; and
apply, with a prediction and learning engine, a convolutional neural network model to the convolutional feature layers to identify and train a pattern of active regions of interest within each convolutional feature layer to predict whether a target subject possesses the pre-identified character trait.

12. The system of claim 11, wherein the computer executable instructions are further configured to cause the processor to:
store the one or more convolutional neural networks in the data store; and
for each pre-defined character trait, extrapolate from the one or more convolutional neural networks, one or more regions of interest correlated to the pre-defined character trait.

13. The system of claim 12, wherein the computer executable instructions are further configured to cause the processor to apply a deep learning algorithm to the one or more convolutional neural networks.

14. The system of claim 11, wherein the computer executable instructions are further configured to cause the processor to obtain, from the user interface, an indication as to whether the target subject possesses the pre-identified character trait.

15. The system of claim 14, wherein the computer executable instructions are further configured to cause the processor to generate an error signal if the prediction as to whether the target subject possesses the pre-identified character trait does not match the indication from the user interface.

16. The system of claim 15, wherein the computer executable instructions are further configured to cause the processor to tune the convolutional neural network model by applying the error signal to the convolutional neural network model.

17. The system of claim 16, wherein the computer executable instructions are further configured to cause the processor to adjust a set of probabilistic weightings for one or more convolutional layers, wherein a probabilistic weighting indicates a likelihood that the convolutional layer is included in the convolutional neural network model in relation to a corresponding pre-defined character trait.

18. The system of claim 11, wherein the computer executable instructions are further configured to cause the processor to apply a convolutional neural network algorithm to the convolutional feature layers to identify a pattern of active regions of interest within each convolutional feature layer to render a medical diagnosis.

19. The system of claim 18, wherein the computer executable instructions are further configured to cause the processor to store a plurality of convolutional neural networks, each convolutional neural network comprising a set of convolutional feature layers and one or more corresponding medical diagnoses; and
for each medical diagnosis, extrapolating from the plurality of convolutional neural networks, one or more regions of interest correlated to the medical diagnosis.

20. The system of claim 11, wherein the image data source comprises a still camera, video camera, an infrared camera, a 3D point cloud source, a laser scanner, a CAT scanner, a MRI scanner, or an ultrasound scanner.
